(19) United States
(12) Patent Application Publication
(10) Pub. No.: US 2024/0369539 A1
(43) Pub. Date: Nov. 7, 2024

(54) AN ADHERENT CELL CULTURE METHOD FOR GENERATING TIGHT JUNCTION BETWEEN CELLS AND ITS PRODUCT APPLICATION

(71) Applicant: ZHEJIANG UNIVERSITY, Hangzhou (CN)

(72) Inventors: Yong HE, Hangzhou (CN); Nian LIU, Hangzhou (CN); Jianzhong FU, Hangzhou (CN)

(21) Appl. No.: 18/249,339

(22) PCT Filed: Oct. 28, 2022
(86) PCT No.: PCT/CN2022/128137

§ 371 Date: Apr. 17, 2023

(30) Foreign Application Priority Data
Nov. 29, 2021 (CN) 202111433970.7
Mar. 14, 2022 (CN) 202210245504.4

(51) Int. Cl. G01N 33/50; C12N 5/071

(52) U.S. Cl. CPC G01N 33/5082; C12N 5/0697; C12N 2513/00; C12N 2533/74; C12N 2537/10

(57) ABSTRACT

The present disclosure provides a culture method of adherent cells generating a tight junction structure and a product thereof. The culture method includes: inoculating adherent cells resuspended in a culture medium onto a bottom support liquid in a culture container for culture to obtain a membranous cell sheet, where the bottom support liquid has higher density than the culture medium, and is not miscible with the culture medium. According to the culture method in the present disclosure, the bottom support liquid is applied to the culture of the adherent cells for the first time, the formation of a tight junction between the adherent cells and the secretion of a large number of extracellular matrices can be promoted so as to form a membranous cell sheet having high cell density, and in vivo tissue structures are well simulated.

### CROSS-REFERENCE TO RELATED APPLICATIONS

This application is a 371 of international Patent Application No. PCT/CN2022/128137 filed Oct. 28, 2022, which claims priority to CN 202111433970.7, filed Nov. 29, 2021 and CN 202210245504.4, filed Mar. 14, 2022. The contents of each of the above-captioned patent applications are hereby expressly incorporated herein by reference in their entireties.

### TECHNICAL FIELD

The present disclosure belongs to the technical field of tissue engineering and biological manufacturing subordinate to biomedical engineering, and specifically relates to a culture method of adherent cells generating a tight junction structure and application of a product thereof.

### BACKGROUND

The development of technologies for maintenance and growth of cells in vitro is an important milestone in bioscience. In vitro cell culture refers to the process of providing necessary physical and chemical cues and growth factors for cells which are obtained in vitro and originally grow in vivo so as to maintain a basic life process of the cells. The cell culture is an important part of the research in life science and clinical medicine. The basic life process of the human body can be conveniently reproduced in vitro, convenience is provided for studying the formation and functions of human tissues or organs in vitro, and a convenient and alternative way is provided for studying pathophysiological processes, disease occurrence and drug development.

In order to conveniently reproduce the life process of cells with high throughput, cell culture based on a solid matrix has become a most widely used cell culture method. The cell culture method based on a solid matrix is simple, low in cost and high in robustness, and has never undergone major changes since the introduction in 1912.

However, according to a traditional cell culture method based on a solid matrix, a cell microenvironment cannot be reconstructed in vivo, so that cell phenotypes and genotypes are changed, and the reliability and reproducibility of the research in life science and clinical medicine are affected. According to some advanced cell culture methods based on a solid matrix, a hydrogel similar to an extracellular matrix is used as a support for cell culture, so that a suitable three-dimensional microenvironment can be provided for cells. However, it is difficult to ensure that cells cultured by using a three-dimensional solid matrix has high cell density comparable to that of the human tissues. In addition, due to empirical dependence and high cost, wide application of a cell culture method based on a three-dimensional solid matrix is also limited. By assembling monodisperse cells into cell microspheres using magnetic fields, sound fields or mechanical forces, cells having high cell density and a tight junction comparable to those of the human tissues can be constructed. However, due to complex operation and high learning threshold, a cell culture method based on an external force mediated solid matrix has not been widely used.

Therefore, it is necessary to provide a cell culture method which is easy to use and capable of promoting the formation of a tight junction between adherent cells and the secretion of a large number of extracellular matrices so as to form cells having high cell density.

A blood-brain barrier refers to a “barrier” between blood vessels and the brain that selectively prevents certain substances from entering the brain through the blood vessels. The blood-brain barrier is essentially a complex cell or tissue structure between peripheral blood and brain tissues, which controls the passing and exchange of substances between blood and cerebrospinal fluid to regulate and ensure the homeostasis of the environment in the brain. Cells constituting the blood-brain barrier are mainly endothelial cells. The endothelial cells form a tight junction structure under the action of various tight junction proteins and interact with cells such as glial cells and pericytes to form a special barrier system, namely the blood-brain barrier.

Due to low permeability, the blood-brain barrier has become a natural barrier to the delivery of drugs into the brain, so that the drugs are prevented from being effectively delivered into the brain. Therefore, effective permeation of the drugs into the blood-brain barrier should be taken into account during the research and development of the delivery of the drugs into the brain. In addition, the homeostasis of the microenvironment of the brain can be disrupted by abnormal development and function loss of the blood-brain barrier, leading to dysfunction of the nervous system, so as to cause stroke, Alzheimer's disease, Parkinson's disease and many other nervous system diseases. Therefore, the development and in-depth study of an in vitro blood-brain barrier model will provide an important theoretical basis for drug development and disease diagnosis and treatment of nervous system diseases.

Existing blood-brain barrier models mainly include an in vivo animal model, an in vitro model based on a microfluidic chip technology and a model based on a Transwell chamber. According to the in vivo animal model, a blood-brain barrier of mice, rats, rabbits and other animals is mostly used for study. Due to species differences between small animals and the human body, the accuracy of a blood-brain barrier model is difficult to be guaranteed. According to the in vitro model based on a microfluidic chip, a microenvironment of a human blood-brain barrier can be well reproduced. However, due to difficult operation and high technical threshold, wide application of the model based on a microfluidic chip is hindered. The in vitro model based on a Transwell chamber has the characteristic of being convenient to operate, and the species differences can be minimized as much as possible by using human cells. However, it is difficult for the common model based on a Transwell chamber to well promote the formation of a tight junction structure between cells, so that the effectiveness of a blood-brain barrier is difficult to be guaranteed.

Therefore, it is necessary to provide an effective blood-brain barrier model that is simple to operate, low in technical threshold and capable of promoting the formation of a tight junction between cells.

### SUMMARY

In order to solve the problems in the prior art, the present disclosure provides a culture method of adherent cells generating a tight junction structure. The culture method has the characteristics of simple and easy operation and low additional cost, and can promote the formation of a tight junction between adherent cells and the secretion of a large number of extracellular matrices so as to form a membranous cell sheet having high cell density.

According to the culture method of adherent cells generating a tight junction structure provided by the present disclosure, limitations of traditional cell culture methods to simple and easy operation and low additional cost as well as good reconstruction of a cell microenvironment in vivo are broken through, and a great commercial value is achieved.

A culture method of adherent cells generating a tight junction structure includes: inoculating adherent cells resuspended in a culture medium onto a bottom support liquid in a culture container, and completing culture on the surface of the bottom support liquid,
where the bottom support liquid has higher density than the culture medium, and is not miscible with the culture medium.

The adherent cells are growing cells attached to a wall during cell culture. The culture method can be applied to the culture of normal adherent cells and the culture of a membranous cell sheet having high cell density. When the culture method is applied to the formation of a membranous cell sheet, the membranous cell sheet obtained can be applied to 3D bioprinting, cell patching and drug testing.

As a preference, the bottom support liquid includes, but is not limited to, a mixture of one or more of fluorinated oil, fluoroalkane compounds, siloxane compounds (such as silicone oil and uncured polydimethylsiloxane), and ester compounds (such as dimethyl carbonate and dimethyl sulfate).

As a further preference, the bottom support liquid is a mixture of one or more of 3M Novec HFE series fluorinated oil (such as HFE7500), 3M Fluorinert FC series fluorinated oil, TECCEM Fluoronox series fluorinated oil, silicone oil, uncured polydimethylsiloxane, dimethyl carbonate, and dimethyl sulfate.

As a preference, the bottom support liquid in the culture container is added in an amount of greater than 0.08 mL/cm². As a further preference, the bottom support liquid is added in an amount of 0.3-0.7 mL/cm².

As a preference, the adherent cells are inoculated in a concentration of 2*10⁴-2*10⁸ pcs/cm². Further preferably, the concentration is 1*10⁶-2*10⁶ pcs/cm².

As a preference, the adherent cells are cultured at a temperature of 35-39° C. for 1-28 days. Further preferably, the adherent cells are cultured at a temperature of 37° C. for 1-14 days.

As a preference, after inoculation, the adherent cells are cultured in an incubator with a carbon dioxide concentration of 5%.

As a preference, the adherent cells are frozen cells after recovery or cells after passage digestion.

As a preference, the adherent cells are one or more of stem cells, tumor cells, epithelial cells, endothelial cells, glial cells, pericytes, fibroblasts, nerve cells, smooth muscle cells, skeletal muscle cells, cardiomyocytes, liver cells, bile duct cells, stellate cells, bone derived cells, immune related cells, and various other tissue or organ derived cells.

As a preference, the culture medium is replaced once every 10-15 hours during the culture, and the volume of the replaced culture medium is 70-90% of that of the original culture medium. As a further preference, the culture medium is replaced once every 12 hours during the culture.

As a further preference, when the culture medium is replaced, the new culture medium is preheated before replacement.

As a preference, the bottom support liquid is sterilized and then added to the culture container.

As a further preference, the bottom support liquid is sterilized by one or more sterilization methods of chemical reagent sterilization, ray sterilization, dry heat sterilization, moist heat sterilization, and filtration sterilization. More further preferably, ultraviolet sterilization is used.

The culture container may be a culture plate and a culture dish, and may also be any container suitable for common cell culture. The culture container may also be a container with a customized material, shape, and structure. As a preference, the culture container is a commercial culture plate.

As a specific preference, a culture method of adherent cells generating a tight junction structure includes the following steps:
step one: sterilizing a bottom support liquid, and adding the sterilized bottom support liquid to a culture container of adherent cells in advance;
step two: resuspending the adherent cells in a culture medium, and seeding the resuspended adherent cells onto the bottom support liquid; and
step three: culturing the inoculated adherent cells at a temperature of 35-39° C. for 1-28 days, and replacing the culture medium every 10-15 hours during the culture.

When the culture method of adherent cells generating a tight junction structure in the present disclosure is applied to the research in life science and clinical medicine, the culture of the adherent cells can be achieved, and the formation of a tight junction between the adherent cells and the secretion of a large number of extracellular matrices can be promoted so as to form a membranous cell sheet.

According to the culture method of adherent cells generating a tight junction structure in the present disclosure, the bottom support liquid is applied to cell culture for the first time, a more diversified cell culture mode is achieved, and the matrix type in existing cell culture methods is expanded.

The present disclosure further provides a membranous cell sheet having a tight junction structure prepared by the method above.

In order to solve the problems in the prior art, the present disclosure provides an in vitro blood-brain barrier model having a tight junction structure. The blood-brain barrier model can effectively simulate functions of an in vivo blood-brain barrier, and has the characteristics of simple operation and low technical threshold. A great commercial value is achieved.

The present disclosure further provides application of the in vitro blood-brain barrier model having a tight junction structure to the research and development of blood-brain barrier related drugs.

An in vitro blood-brain barrier model having a tight junction structure includes:
a bracket that fits the culture plate;
a mesh substrate support for sealing the bottom of the bracket in the culture plate;
a membranous cell sheet that is loaded on the mesh substrate support and has a tight junction structure;
and a hydrogel bulk for encapsulating the membranous cell sheet, the mesh substrate support and the bracket as a whole.

The membranous cell sheet having a tight junction structure can be obtained by the culture method of adherent cells generating a tight junction structure according to any one of the foregoing descriptions.

After the in vitro blood-brain barrier model having a tight junction structure is completely constructed, the lower end (membranous cell sheet) is required to be placed in a cell culture medium to maintain cell activity. The cross section of the bracket is round, square, rectangular, triangular or special-shaped.

The membranous cell sheet consists of one or more types of blood-brain barrier related cells, and can be used for simulating a tight junction structure between blood-brain barrier cells.

As a preference, the bracket is of a round tube structure. In order to facilitate processing and mounting and improve the sealing effect of the blood-brain barrier model, the mesh substrate support is of a round sheet structure matching with the round tube structure of the bracket.

As a preference, the upper end of the bracket is provided with a positioning part for positioning the bracket. The positioning part is used for positioning the blood-brain barrier model, so that the lower end of the bracket loaded with the membranous cell sheet can be suspended in the cell culture medium. In this case, the membranous cell sheet can separate the cell culture medium into an inner part and an outer part of the blood-brain barrier model, which are used for simulating an inner part and an outer part of a blood-brain barrier, so as to reproduce physiological functions of the blood-brain barrier in vitro. The inner part (cell culture medium) of the blood-brain barrier model is used as an upper chamber, and the outer part (cell culture medium) is used as a lower chamber.

As a further preference, the positioning part is a positioning rod that is perpendicular to the central axis of the bracket and is fixedly connected with the upper end of the bracket. When the bracket is placed in a container containing a cell culture medium, the outer end of the positioning rod (the end not connected with the bracket) is placed on the top of the container. The size of the bracket is set according to the size of the container.

As a more further preference, three or more positioning rods are arranged, and the three or more positioning rods are circumferentially and uniformly distributed along the upper end of the bracket.

In order to improve the structural stability of the positioning part, as a more further preference, the positioning part further includes a positioning ring sleeved on the outside of the bracket, the positioning rod is separately connected with the upper end of the bracket and the positioning ring, and the radius of the positioning ring is less than the sum of the radius of the bracket and the length of the positioning rod.

As a preference, the lower end of the bracket is provided with a supporting part for mounting the substrate support. The supporting part is an annular mounting table arranged along the inner wall of the lower end of the bracket, and the inner diameter of the mounting table is greater than the outer diameter of the substrate support.

As a preference, the mesh substrate support is a polycaprolactone (PCL) support obtained by high-precision near-field direct-writing 3D printing, where the distance between two adjacent filaments in the same direction is 0.1-10 mm. Further preferably, the distance is 0.8-1.2 mm.

As a preference, the hydrogel structure is prepared from a substance that is converted from a liquid state to a gel state after being stimulated by external conditions (such as temperature and light).

As a further preference, the hydrogel structure is prepared from one or more of gelatin, gelatin derivatives, hyaluronic acid, hyaluronic acid derivatives, alginate compounds, Pluronic F-127, fibrinogen, collagen, silk fibroin, chitosan, agarose, polyethylene glycol, and polyethylene oxide. More further preferably, methacrylic anhydride gelatin (GelMA) is used.

As a preference, a culture method of the membranous cell sheet having a tight junction structure includes:
inoculating cells resuspended in a culture medium onto a bottom support liquid in a culture container, and completing culture on the surface of the bottom support liquid to form the membranous cell sheet having a tight junction structure,
where the bottom support liquid has higher density than the culture medium, and is not miscible with the culture medium.

According to the culture method of the membranous cell sheet, the bottom support liquid is used as a support for the growth of the cells. When a liquid matrix is used for culture, extracellular matrices secreted by the cells cannot be attached to the liquid matrix, but can only be attached to the other adjacent cells, so that more extracellular matrices are secreted by the cells, and a membranous cell sheet having high cell density and a tight junction is formed.

As a further preference, the bottom support liquid includes, but is not limited to, a mixture of one or more of fluorinated oil, fluoroalkane compounds, siloxane compounds (such as silicone oil and uncured polydimethylsiloxane), and ester compounds (such as dimethyl carbonate and dimethyl sulfate).

The main factor of choosing a hydrophobic liquid represented by the fluorinated oil as the bottom support liquid is that due to a hydrophobic effect of the fluorinated oil as a liquid matrix, radial inward surface tension from all aspects will be provided for the cells. The shape of the cells is controlled by a Young-Laplace equation. That is to say, potential energy is minimized under the constraint of the conservation of the volume of each cell, so that the cells become stereospheres, and the stereosphere cells are spontaneously self-assembled to form a hexagonal tight junction structure.

As a more further preference, the bottom support liquid is a mixture of one or more of 3M Novec HFE series fluorinated oil (such as HFE7500), 3M Fluorinert FC series fluorinated oil, TECCEM Fluoronox series fluorinated oil, silicone oil, uncured polydimethylsiloxane, dimethyl carbonate, and dimethyl sulfate.

As a further preference, the bottom support liquid in the culture container is added in an amount of greater than 0.08 mL/cm². As a more further preference, the bottom support liquid is added in an amount of 0.3-0.7 mL/cm².

As a further preference, the cells are frozen cells after recovery or cells after passage digestion.

As a further preference, the cells are blood-brain barrier model related cells, including but not limited to one or more of endothelial cells, glial cells, and pericytes. More further preferably, endothelial cells are used.

As a further preference, the cells are inoculated in a concentration of 2*10⁴-2*10⁸ pcs/cm² during cell culture. More further preferably, the concentration is 1*10⁶-2*10⁶ pcs/cm².

As a further preference, the cells are cultured at a temperature of 35-39° C. for 1-28 days. More further preferably, the cells are cultured at a temperature of 37° C. for 1-14 days, and the cultured cells form a membranous cell sheet having a tight junction structure.

As a further preference, after inoculation, the cells are cultured in an incubator with a carbon dioxide concentration of 5%.

As a further preference, the culture medium is replaced once every 10-15 hours during the cell culture, and the volume of the replaced cell culture medium is 70-90% of that of the original cell culture medium. More further preferably, the culture medium is replaced once every 12 hours during the culture.

As a more further preference, when the culture medium is replaced, the new culture medium is preheated before replacement.

As a further preference, the bottom support liquid is sterilized and then added to the culture container.

As a further preference, the bottom support liquid is sterilized by one or more sterilization methods of chemical reagent sterilization, ray sterilization, dry heat sterilization, moist heat sterilization, and filtration sterilization. More further preferably, ultraviolet sterilization is used.

The culture container may be a culture plate and a culture dish, and may also be any container suitable for common cell culture. The culture container may also be a container with a customized material, shape, and structure. As a preference, the culture container is a commercial culture plate.

As a further preference, the mesh substrate support is buried in the bottom support liquid first, and then the cells are inoculated. After the membranous cell sheet is formed at the end of cell culture, the mesh substrate support is lifted upward and removed, whereby the membranous cell sheet is attached to the substrate support, and the mesh substrate support loaded with the membranous cell sheet is obtained. Since the membranous cell sheet formed is relatively fragile, it is difficult to remove the membranous cell sheet completely without the help of external forces. According to the technical solutions, the mesh substrate support is used as a supporting structure for the membranous cell sheet. The membranous cell sheet is completely removed under the support of the substrate support, and the mesh substrate support and the membranous cell sheet are applied to the blood-brain barrier model as a whole.

In order to facilitate the removal of the substrate support, as a further preference, a collection support may be added as an auxiliary removal and placement tool to remove and place the substrate support. The collection support includes an annular structure at the bottom and a lifting rod connected with the annular structure. The inner side of the annular structure is provided with an annular boss for mounting the substrate support.

The lifting rod is parallel to the central axis of the annular structure, the lower end of the lifting rod is connected with the annular structure, and the upper end of the lifting rod is provided with a bent lifting handle. The lifting handle can be hung on the top of the culture container when the collection support is placed in the culture container. The size of the collection support is set according to the size of the culture container.

During use, the mesh substrate support is mounted on the collection support first and then placed in the bottom support liquid. When the membranous cell sheet is formed and removed, the collection support is directly removed, and then the mesh substrate support and the membranous cell sheet are removed from the collection support.

As a further preference, the collection support is a polylactic acid (PLA) support obtained by 3D printing.

As a preference, the mesh substrate support and the collection support are sterilized and then added to the culture container. As a further preference, one or more sterilization methods of chemical reagent sterilization, ray sterilization, dry heat sterilization, moist heat sterilization, and filtration sterilization are used for sterilization. More further preferably, ultraviolet sterilization is used.

As a preference, the mesh substrate support loaded with the membranous cell sheet is removed from the collection support and then mounted on the bracket. The mesh substrate support loaded with the membranous cell sheet is suspended in the container containing the cell culture medium through the positioning part of the bracket, so as to obtain the in vitro blood-brain barrier model.

With cell culture in a 12-well culture plate as an example, a construction process of the in vitro blood-brain barrier model having a tight junction structure is as follows.

(1) Preparation of a membranous cell sheet having a tight junction structure

A clean and sterilized 12-well culture plate is prepared, and a clean and sterilized collection support with a customized size and a mesh substrate support (the mesh substrate support is mounted on the collection support) are placed in the culture plate in sequence.

The outer diameter of the collection support with a customized size is slightly less than the inner diameter of the 12-well culture plate; and the height is slightly greater than the depth of the 12-well culture plate.

A bottom support liquid (with the liquid level at least higher than the substrate support) and a cell culture medium containing cells are added to the 12-well culture plate in sequence for culture. After the culture is completed, a membranous cell sheet having a tight junction structure is formed on the surface of bottom support liquid,
where the bottom support liquid has higher density than the culture medium, and is not miscible with the culture medium.

### (2) Construction of an In Vitro Blood-Brain Barrier Model

Another clean and sterilized 6-well culture plate is prepared. The collection support is slowly removed from the 12-well culture plate, whereby the membranous cell sheet is attached to the substrate support. The mesh substrate support loaded with the membranous cell sheet is separated from the collection support and placed in a clean and sterilized bracket with a customized size.

The bracket is suspended on an orifice plate, and the spacing between the bottom of the bracket (namely the membranous cell sheet) and the orifice plate is 1-10 mm.

The mesh substrate support loaded with the membranous cell sheet and the bracket are encapsulated with a hydrogel, and the encapsulated bracket is placed in the 6-well culture plate added with a cell culture medium for culture, so as to maintain cell activity.

As a preference, the collection support is a polylactic acid (PLA) support obtained by 3D printing, and has an outer diameter of 21.2 mm and a height of 19 mm.

As a preference, the mesh substrate support is of a round mesh structure, and has a diameter of 15-21 mm and a filament spacing of 0.1-10 mm.

As a preference, the mesh substrate support is a polycaprolactone (PCL) support obtained by high-precision near-field direct-writing 3D printing, and has a diameter of 19 mm and a filament spacing of 1 mm.

As a preference, the bracket is a polylactic acid (PLA) support obtained by 3D printing, and has an outer diameter of 35 mm and a height of 17 mm, and the spacing between the bottom of the bracket and the orifice plate is 5 mm.

The in vitro blood-brain barrier model having a tight junction structure according to any one of the foregoing descriptions is applied to the research and development of blood-brain barrier related drugs.

As a preference, the bottom (membranous cell sheet) of the blood-brain barrier model is suspended in a cell culture medium, a blood-brain barrier related drug is added to the cell culture medium (upper chamber) inside the blood-brain barrier model, and the concentration of the drug in the cell culture medium (lower chamber) outside the blood-brain barrier model is detected so as to determine a permeation effect of the drug in the blood-brain barrier model.

According to a construction method of an in vitro blood-brain barrier model having a tight junction structure in the present disclosure, blood-brain barrier related endothelial cells are prepared into a membranous cell sheet having a tight junction structure, and the membranous cell sheet, an bracket with a customized size and a mesh substrate support are encapsulated as a whole with a hydrogel, and the bracket is separated into an upper part and a lower part which are used for simulating an inner part and an outer part of a blood-brain barrier, so that physiological functions of the blood-brain barrier can be reproduced in vitro, and the permeability of a drug in the blood-brain barrier is observed.

According to a construction method of an in vitro blood-brain barrier model having a tight junction structure in the present disclosure, a platform for simulating an in vivo blood-brain barrier is constructed. The platform can be applied to the research in life science and clinical medicine, and an important theoretical basis is provided for drug development and disease diagnosis and treatment of nervous system diseases.

Certainly, the membranous cell sheet structure having a tight junction structure can also be prepared separately in the present disclosure, and after the preparation is completed, the membranous cell sheet having a tight junction structure is placed on the substrate support.

The present disclosure provides a method for constructing or preparing an in vitro blood-brain barrier model having a tight junction structure. The method includes the following steps: (1) culturing a membranous cell sheet by a culture method of adherent cells generating a tight junction structure; (2) printing a mesh substrate support of the membranous cell sheet; (3) printing an bracket with a customized size; and (4) encapsulating the membranous cell sheet, the mesh substrate support and the bracket with a hydrogel.

In the above step (1), the culturing can be conducted by the culture method of adherent cells generating a tight junction structure according to any one of the foregoing technical solutions.

The membranous cell sheet consists of one or more types of blood-brain barrier related cells, and can be used for simulating a tight junction structure between blood-brain barrier cells. The mesh substrate support of the membranous cell sheet is used for extracting the membranous cell sheet and providing mechanical support for the membranous cell sheet. The bracket can be suspended in different types of orifice plates to separate the orifice plate into an upper chamber and a lower chamber. According to the present disclosure, the construction, characterization, barrier function, drug evaluation and other functions of the in vitro blood-brain barrier model are integrated into one. The method can be used for the in vitro simulation of a blood-brain barrier model and the testing application of blood-brain barrier related drugs. Compared with existing blood-brain barrier models, the present disclosure has the advantages that the contradiction between the accuracy and simplicity of the construction of the in vitro blood-brain barrier model is solved, the in vivo real environment is getting closer, the experimental efficiency is improved, and a good application prospect in the research and drug development of blood-brain barrier related diseases is achieved.

Compared with the prior art, the present disclosure has the following beneficial effects.

(1) The culture method of adherent cells generating a tight junction structure in the present disclosure is a supplementary method to existing cell culture methods, the matrix type of the existing cell culture methods is expanded, and the culture method is simple and easy to operate and low in additional cost and has a great commercial value.

(2) According to the culture method of adherent cells generating a tight junction structure in the present disclosure, the formation of a tight junction between the adherent cells and the secretion of a large number of extracellular matrices can be promoted so as to form a membranous cell sheet having high cell density. In vivo tissue structures are well simulated, and the method is more applicable to the research in life science and clinical medicine and has a great application prospect.

(3) According to the culture method of adherent cells generating a tight junction structure in the present disclosure, the steps of cleaning with a phosphate buffer and digestion with a pancreatic enzyme in traditional adherent cell culture methods can be omitted, so that extracellular matrices in a growth process of the adherent cells are retained, a cell growth microenvironment is better restored, and the research and application in life science and clinical medicine are facilitated.

(4) According to the culture method of adherent cells generating a tight junction structure in the present disclosure, the whole culture medium can be maintained in a dynamic horizontal state in the culture process, so that uniform distribution of the cells in the culture process is facilitated.

(5) Compared with an in vivo blood-brain barrier model of animals, the blood-brain barrier model of the present disclosure has the advantages that species differences are eliminated, experimental data obtained through the platform (blood-brain barrier model) are more consistent with real situations of the human body, and the accuracy of the development of blood-brain barrier drugs is improved.

(6) Compared with an in vitro blood brain barrier model based on a microfluidic chip, the blood-brain barrier model of the present disclosure has the advantages that the operation difficulty and the technical threshold are reduced. On the premise of ensuring the effectiveness and accuracy of the blood-brain barrier model, the blood-brain barrier model has the characteristics of being simple to operate and easy to popularize, and has a great commercial value.

(7) Compared with an in vitro common blood-brain barrier model based on a Transwell chamber, the blood-brain barrier model of the present disclosure has the advantages that the formation of a tight junction structure between cells is promoted, the blood-brain barrier model has a tissue structure similar to real situations of human tissues, and an effective blood-brain barrier model is formed.

(8) A blood-brain barrier similar to that under physiological conditions in the tissue structure, transmembrane resistance value, permeability, toxic drug response and other aspects is constructed in vitro for the first time in the present disclosure, and an important research platform is provided for drug development and disease diagnosis and treatment of nervous system diseases.

### BRIEF DESCRIPTION OF DRAWINGS

FIG. 1 is a schematic diagram showing the process of forming a membranous cell sheet on a 12-well culture plate in an embodiment of the present disclosure, where 101 refers to a 12-well culture plate, 102 refers to a bottom support liquid, 103 refers to frozen cells after recovery, 104 refers to a culture medium, and 105 refers to a membranous cell sheet.

FIG. 2 is a schematic diagram showing the comparison between a culture method of adherent cells generating a tight junction in an embodiment of the present disclosure and a traditional adherent cell. culture method, where FIG. 2A is a schematic diagram showing the flow of the culture method of adherent cells generating a tight junction structure; and FIG. 2B is a schematic diagram showing the flow of the traditional adherent cell culture method.

FIG. 3 shows physical images of a membranous cell sheet formed by cells in a 12-well culture plate in an embodiment of the present disclosure, where FIG. 3A is a lateral upward view of the membranous cell sheet formed by cells in a 12-well culture plate in an embodiment of the present disclosure, and the membranous cell sheet formed by adherent cells generating a tight junction structure is as shown in a circle of the dotted line; and FIG. 3B is a top view of the membranous cell sheet formed by cells in a 12-well culture plate in an embodiment of the present disclosure.

FIG. 4 shows local electron microscopy images of a membranous cell sheet cultured for 3 days in an embodiment of the present disclosure, where FIG. 4A is a local electron microscopy image of the membranous cell sheet magnified by 400 times; FIG. 4B is a local electron microscopy image of the membranous cell sheet magnified by 2,000 times; FIG. 4C is a local electron microscopy image of the membranous cell sheet magnified by 5,000 times; and FIG. 4D is a local electron microscopy image of the membranous cell sheet magnified by 8,000 times.

In FIG. 5, FIG. A is a diagram showing the comparison of the vitality of cells in a bottom support liquid under different traditional adherent cell culture conditions; and FIG. B shows detection results of the living/dead cell percentage of cells in a bottom support liquid in an embodiment of the present disclosure.

FIG. 6 shows physical images of membranous cell sheets formed in cell culture containers prepared from different materials by adopting a culture method in an embodiment of the present disclosure.

FIG. 7 shows physical images of a membranous cell sheet formed in a cell culture container with a customized shape by adopting a culture method in an embodiment of the present disclosure, where FIG. 7A is a top view of the membranous cell sheet formed in a cell culture container with a customized shape by adopting a culture method in an embodiment of the present disclosure; and FIG. 7B is a side view of the membranous cell sheet formed in a cell culture container with a customized shape by adopting a culture method in an embodiment of the present disclosure, and a bottom support liquid is as shown in a square circle of the dotted line.

FIG. 8 is a physical image of a membranous cell sheet formed in a cell culture container with a customized structure by adopting a culture method in an embodiment of the present disclosure.

FIG. 9 shows micrographs showing growth conditions of different cells under culture conditions in an embodiment of the present disclosure, where FIG. 9A is a micrograph showing the growth conditions of human foreskin fibroblasts (HFF) derived from ectoderm; FIG. 9B is a micrograph showing the growth conditions of human umbilical vein endothelial cells (HUVEC) derived from mesoderm; FIG. 9C is a micrograph showing the growth conditions of hepatocellular carcinoma cells (HepG2) derived from endoderm; and FIG. 9D is a micrograph showing the growth conditions of bone marrow mesenchymal stem cells (BMSC) derived from stem cells.

FIG. 10 is a schematic diagram showing the processes of preparing a membranous cell sheet having a tight junction structure and constructing an in vitro blood-brain barrier model in an embodiment of the present disclosure, where 1 refers to a clean and sterilized 12-well culture plate, 2 refers to a collection support, 3 refers to a substrate support, 4 refers to a bottom support liquid, 5 refers to cells, 6 refers to a cell culture medium, 7 refers to a clean and sterilized 6-well culture plate, 8 refers to an bracket, and 9 refers to a hydrogel.

FIG. 11 is a schematic diagram showing the structure of an in vitro blood-brain barrier model in an embodiment of the present disclosure.

FIG. 12 is a schematic diagram showing structures of a collection support and an bracket in an embodiment of the present disclosure, where FIG. a in FIG. 12 shows an oblique view, a top view and a side view of the collection support; FIG. b in FIG. 12 shows an oblique view, a top view and a side view of the bracket; and 10 refers to an annular structure, 11 refers to a lifting rod, 12 refers to a lifting handle, 13 refers to an annular boss; 80 refers to a round tube structure, 81 refers to a positioning rod, 82 refers to a positioning ring, and 83 refers to an annular mounting table.

FIG. 13 shows a physical image of a mesh substrate support in an embodiment of the present disclosure and micrographs of magnified edge and center parts.

FIG. 14 shows physical images in processes in an embodiment of the present disclosure, where FIG. a in FIG. 14 is a physical image of a membranous cell sheet having a tight junction structure prepared; FIG. b in FIG. 14 is a physical image (top view) of the membranous cell sheet having a tight junction structure prepared; FIG. c in FIG. 14 is a physical image of the membranous cell sheet having a tight junction structure prepared and collected by a collection support; FIG. d in FIG. 14 is a physical image of the membranous cell sheet on a mesh substrate support after the membranous cell sheet having a tight junction structure is prepared (mesh substrate support loaded with the membranous cell sheet); and FIG. e in FIG. 14 is a physical image of an in vitro blood-brain barrier model.

FIG. 15 shows micrographs and electron microscopy images of a membranous cell sheet having a tight junction structure in an embodiment of the present disclosure, where FIG. a in FIG. 15 is 4-fold micrograph of the membranous cell sheet on a mesh substrate support after the membranous cell sheet having a tight junction structure is obtained; FIG. b in FIG. 15 is a 10-fold micrograph of the membranous cell sheet on a mesh substrate support after the membranous cell sheet having a tight junction structure is obtained; FIG. c in FIG. 15 is a 300-fold micrograph of the membranous cell sheet on a mesh substrate support after the membranous cell sheet having a tight junction structure is obtained; and FIG. d in FIG. 15 is a 4,000-fold micrograph of the membranous cell sheet on a mesh substrate support after the membranous cell sheet having a tight junction structure is obtained.

FIG. 16 shows electron microscopy images of a hydrogel in an embodiment of the present disclosure, where FIG. a in FIG. 16 is a 100-fold electron microscopy image of the surface of the hydrogel; FIG. b in FIG. 16 is a 300-fold electron microscopy image of the surface of the hydrogel surface; FIG. c in FIG. 16 is a 50-fold electron microscopy image of the inside of the hydrogel; and FIG. d in FIG. 16 is a 200-fold electron microscopy image of the inside of the hydrogel.

FIG. 17 shows test results of verifying the tightness of an bracket (blood-brain barrier model) in an embodiment of the present disclosure.

FIG. 18 shows test results of the transmembrane resistance of a blood-brain barrier under different blood-brain barrier construction conditions.

FIG. 19 shows test results of fluorescent substances with different molecular weights passing through a blood-brain barrier under different blood-brain barrier construction conditions.

FIG. 20 shows test results of the permeability of a blood-brain barrier before and after treatment with a blood-brain barrier toxic drug (methamphetamine) in an embodiment of the present disclosure.

FIG. 21 shows test results of blood-brain barrier related drugs (dopamine and L-dopamine) passing through a blood-brain barrier in an embodiment of the present disclosure, where FIG. a in FIG. 21 is a schematic diagram showing the passing of the blood-brain barrier related drugs through the blood-brain barrier; and FIG. b in FIG. 21 shows detection results of the blood-brain barrier related drugs passing through the blood-brain barrier.

### DETAILED DESCRIPTION OF EMBODIMENTS

In order to make the objects, technical solutions and advantages of the present disclosure more clearly understood, the present disclosure is further explained in detail below in conjunction with embodiments. Unless otherwise specified, all equipment and reagents used in various embodiments and test examples can be commercially available. The specific embodiments described herein are intended only to explain the present disclosure, rather than to define the present disclosure.

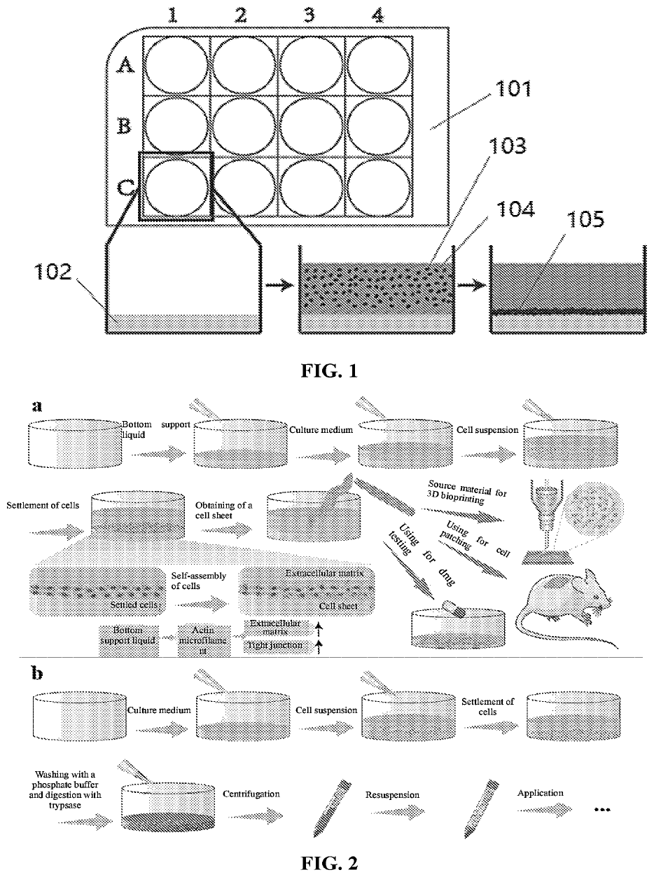

As shown in FIG. 1, a culture method of adherent cells generating a tight junction structure includes the following steps:
1, loading a bottom support liquid 102 (HFE7500) into a transparent centrifugal tube, and conducting ultraviolet sterilization for 2 hours;
2, resuspending frozen cells 105 after recovery in a culture medium 104, and conducting centrifugation at 1,000 rpm for 3 minutes;
3, after a supernatant is removed, resuspending the cells in 1 mL of a culture medium, conducting uniform blowing, and then conducting sampling for cell counting;
4, taking a new 12-well culture plate 101, and adding the bottom support liquid HFE7500 to each well at a rate of 0.5 mL/cm², where since the area of each well is 3.8 cm², 1.9 mL of the bottom support liquid HFE7500 is required for each well;
5, uniformly inoculating a resuspended cell suspension onto the bottom support liquid HFE7500 at a rate of 1*10⁶ pcs/cm², where since the area of each well is 3.8 cm², 3.8*10⁶ cells are required to be inoculated into each well;
6, placing the 12-well culture plate in a carbon dioxide incubator with a carbon dioxide concentration of 5% for culture at 37° C., and allowing the cells to naturally settle to the surface of the bottom support liquid HFE7500 for growth; and
7, during the culture, replacing a preheated cell culture medium once every 12 hours, where the volume of the replaced culture medium is 80% of that of the original culture medium; and finally, forming a membranous cell sheet 105 as shown in FIG. 1.

### Characterization of a Product:

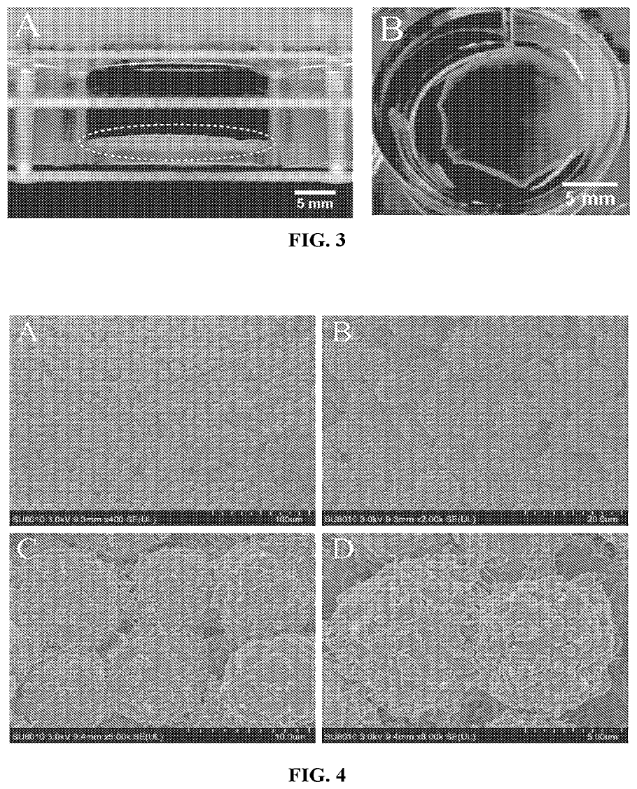

After the above culture, a membranous cell sheet as shown in FIG. 3 can be collected in the incubator. FIG. 3A is a lateral upward view of the membranous cell sheet formed by cells in the 12-well culture plate in the embodiment of the present disclosure, and the membranous cell sheet formed by adherent cells generating a tight junction structure is as shown in a circle of the dotted line; and FIG. 3B is a top view of the membranous cell sheet formed by cells in the 12-well culture plate in the embodiment of the present disclosure. FIG. 4 shows a membranous cell sheet cultured for 3 days photographed under an electron microscope, where FIG. 4A is a local electron microscopy image of the membranous cell sheet magnified by 400 times; FIG. 4B is a local electron microscopy image of the membranous cell sheet magnified by 2,000 times; FIG. 4C is a local electron microscopy image of the membranous cell sheet magnified by 5,000 times; and FIG. 4D is a local electron microscopy image of the membranous cell sheet magnified by 8,000 times. From FIG. 4, it can be seen that the membranous cell sheet is a dense tissue structure formed by a tight junction between cells.

### Functional Tests of a Product:

A CCK8 test is carried out to determine differences in the metabolic activity between a membranous cell sheet collected after culture for 1, 3, and 5 days under culture conditions (bottom support liquid) in the embodiment and cells cultured by a traditional adherent cell culture method under the same culture conditions. Results are as shown in FIG. A in FIG. 5. According to the results as shown in FIG. A in FIG. 5, the membranous cell sheet cultured in the embodiment has no significant differences with the cells cultured by a traditional adherent cell culture method in metabolic activity.

A living/dead cell staining test is carried out to determine the living cell percentage of a membranous cell sheet collected after culture for 1, 3, 5, 7, 14, and 21 days under culture conditions (bottom support liquid) in the embodiment. Results are as shown in FIG. B in FIG. 5. According to the results as shown in FIG. B in FIG. 5, the living cell percentage of the membranous cell sheet cultured in the embodiment can reach 90% or above after culture for 1-21 days.

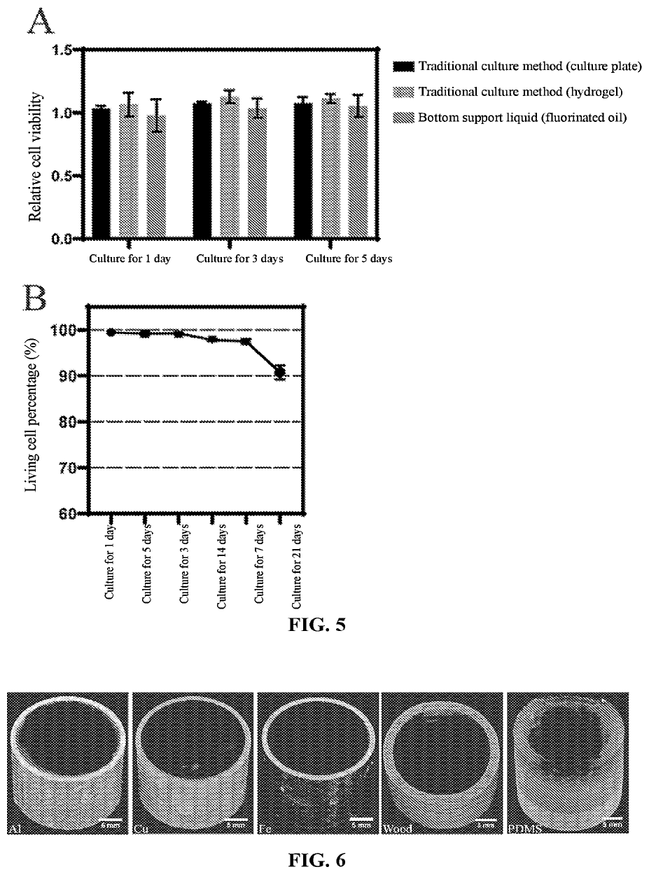

FIG. 6 shows physical images of membranous cell sheets cultured in culture containers prepared from different materials (such as aluminum, copper, iron, wood, and polydimethylsiloxane) by adopting the culture method in the embodiment. From FIG. 6, it can be seen that tight junctions can be achieved between cells so as to form dense membranous cell sheets in culture containers prepared from different materials, indicating that the culture method of adherent cells generating a tight junction structure provided in the embodiment can be applied to the culture containers prepared from various materials, and the membranous cell sheets having high cell density can be cultured. The problem is solved that only a PS (polystyrene) material after special treatment can be used for cell culture by adopting a traditional adherent cell culture method.

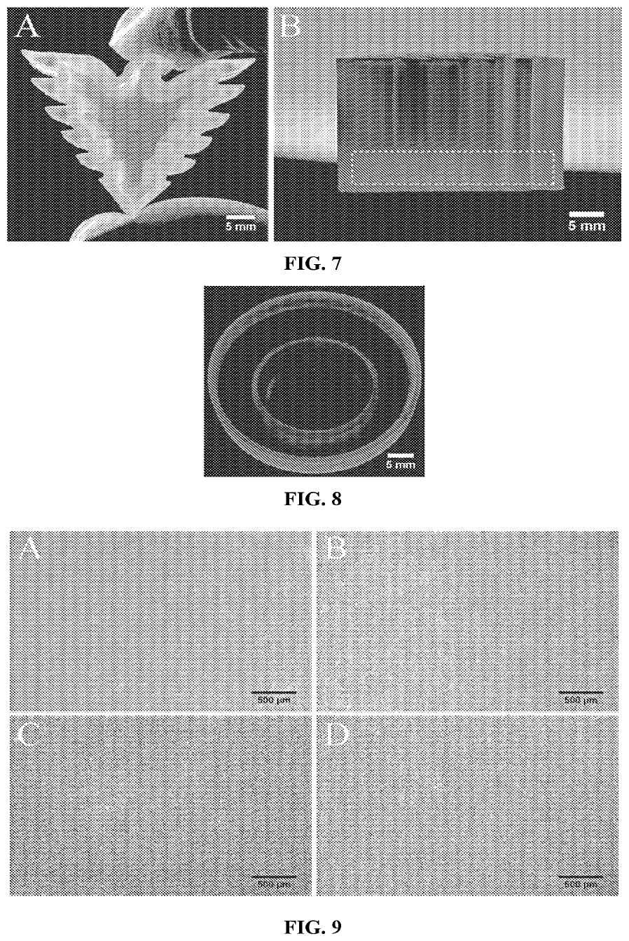

FIG. 7 shows physical images of adherent cells cultured in a culture container with a customized shape by adopting the culture method in the embodiment. FIG. 7A is a top view of a membranous cell sheet formed in a cell culture container with a customized shape by adopting the culture method in the embodiment of the present disclosure; and FIG. 7B is a side view of the membranous cell sheet formed in a cell culture container with a customized shape by adopting the culture method in the embodiment of the present disclosure, and a bottom support liquid is as shown in a square circle of the dotted line. From FIG. 7, it can be seen that a membranous cell sheet having high cell density can be formed successfully by the cells in the culture container with a customized shape.

FIG. 8 is a state image of adherent cells cultured in a culture container with a customized structure by adopting the culture method in the embodiment. From FIG. 8, it can be seen that a membranous cell sheet can be formed successful by the cells in the culture container with a customized structure.

According to the culture results in FIG. 7 and FIG. 8, it is indicated that the culture method of adherent cells generating a tight junction structure in the embodiment can be applied to cell culture containers with customized shapes and structures so as to meet application demands of membranous cell sheets with different shapes and structures.

According to the culture method in the embodiment, 4 typical adherent cells (including human foreskin fibroblasts derived from ectoderm, human umbilical vein endothelial cells derived from mesoderm, hepatocellular carcinoma cells derived from endoderm, and bone marrow mesenchymal stem cells derived from stem cells) are separately selected for cell culture. Culture results are as shown in FIG. 9. From FIG. 9, it can be seen that all the above four kinds of cells can form membranous cell sheets having high cell density after being cultured by adopting the culture method in the embodiment, indicating that the culture method in the embodiment can be applied to culture of a variety of adherent cells and has high applicability.

In summary, fluorinated oil (HFE7500) is used as the bottom support liquid, which can be used for culture of the adherent cells in the embodiment. The culture method has the characteristics of simple and easy operation and low additional cost, and can promote the formation of a tight junction between adherent cells and the secretion of a large number of extracellular matrices so as to form a membranous cell sheet having high cell density. In vivo tissue structures are well simulated, and the method is more applicable to the research in life science and clinical medicine and has a great application prospect and commercial value.

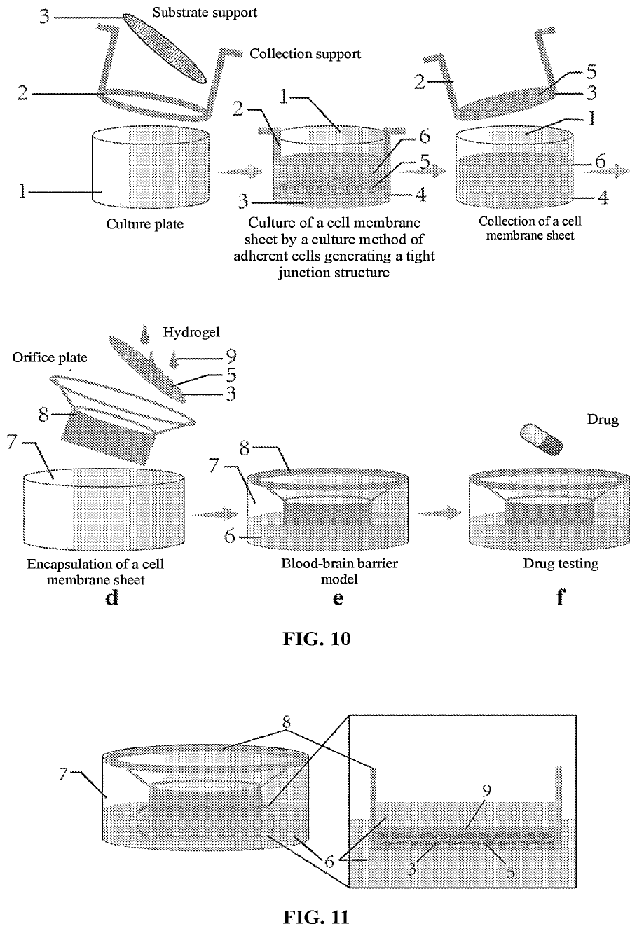

As shown in FIG. 10, a construction method of an in vitro blood-brain barrier model having a tight junction structure includes the following steps:
a, preparing a clean and sterilized 12-well culture plate 1, and placing a clean and sterilized collection support 2 with a customized size and a mesh substrate support 3 (the mesh substrate support is mounted on the collection support) in the culture plate in sequence;
b, adding a bottom support liquid 4 (with the liquid level at least higher than the substrate support) and a cell culture medium 6 containing endothelial cells 5 to the 12-well culture plate in sequence for culture;
c, after the culture is conducted for 24 hours, slowly removing the collection support 2 from the 12-well culture plate, where during the period, a membranous cell sheet is attached to the mesh substrate support 3; and separating the mesh substrate support loaded with the membranous cell sheet from the collection support;
d. preparing another clean and sterilized 6-well culture plate 7, placing the mesh substrate support loaded with the membranous cell sheet in a clean and sterilized bracket 8 with a customized size, and encapsulating the mesh substrate support loaded with the membranous cell sheet and the bracket with a hydrogel 9;
e, placing the encapsulated bracket in the 6-well culture plate added with a cell culture medium for culture, where the membranous cell sheet is suspended in the cell culture medium, and that is to say, both the upper and lower sides of the membranous cell sheet are immersed in the cell culture medium; and
f, constructing an in vitro blood-brain barrier model having a tight junction structure, adding a blood-brain barrier related drug from an upper chamber (the cell culture medium on the upper side of the membranous cell sheet, namely, the cell culture medium in the blood-brain barrier model), and testing a permeation effect of the drug in a blood-brain barrier in a lower chamber (the cell culture medium in the 6-well culture plate).

### Appearance Structure of a Product (Blood-Brain Barrier Model):

Through the above process, an in vitro blood-brain barrier model having a tight junction structure can be constructed, and the structure is mainly as shown in FIG. 11. The bracket is suspended on the 6-well culture plate, and the spacing between the bottom of the bracket 8 and the 6-well culture plate 7 is 5 mm. The mesh substrate support and the membranous cell sheet formed by the endothelial cells are supported at the bottom of the bracket, and the three parts are encapsulated with the hydrogel.

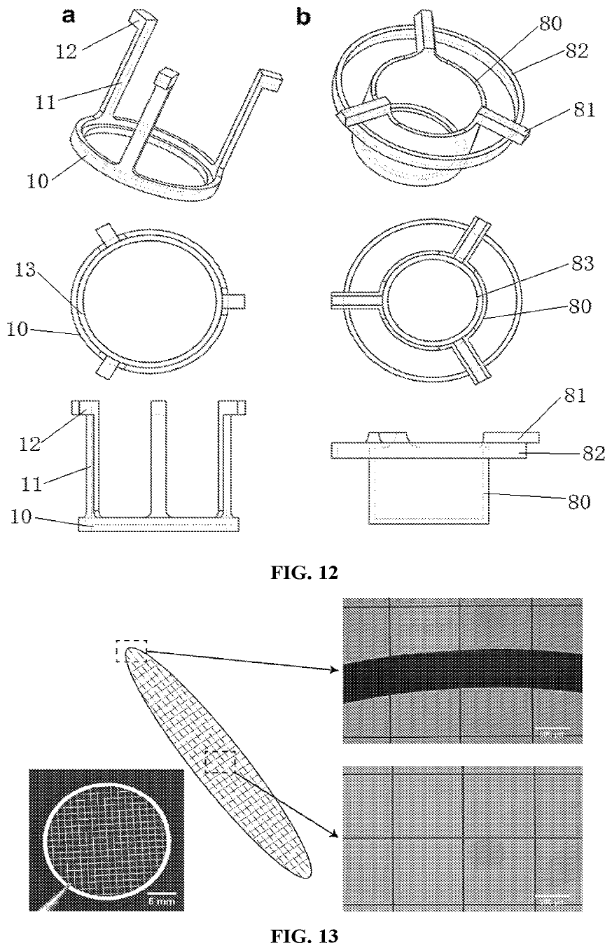

The structure of the collection support is as shown in FIG. a in FIG. 12. The collection support includes an annular structure 10 at the bottom and a lifting rod 11 connected with the annular structure 10. The inner side of the annular structure 10 is provided with an annular boss 13 for mounting the substrate support. The lifting rod 11 is parallel to the central axis of the annular structure 10, the lower end of the lifting rod is connected with the annular structure 10, and the upper end of the lifting rod is provided with a bent lifting handle 12. The lifting handle can be hung on the top of the culture container when the collection support is placed in the culture container.

The structure of the bracket is shown in FIG. b in FIG. 12. The bracket is of a round tube structure 80. The upper end of the round tube structure 80 is sleeved with a positioning ring 82 and three positioning rods 81 for fixedly connecting the upper end of the round tube structure 80 with the positioning ring 82. The positioning rods 81 are perpendicular to the central axis of the round tube structure 80. The three positioning rods 81 are circumferentially and uniformly arranged along the round tube structure 80, and the radius of the positioning ring 82 is less than the sum of the radius of the round tube structure 80 and the length of the positioning rods 81. The inner wall of the lower end of the round tube structure 80 is provided with an annular mounting table 83 for mounting the substrate support. The mesh substrate support loaded with the membranous cell sheet mounted on the annular mounting table 83 is suspended in the cell culture medium by a positioning part consisting of the positioning ring 82 and the three positioning rods 81.

The structure and micrograph of the mesh substrate support are as shown in FIG. 13. The mesh substrate support is of a round mesh structure.

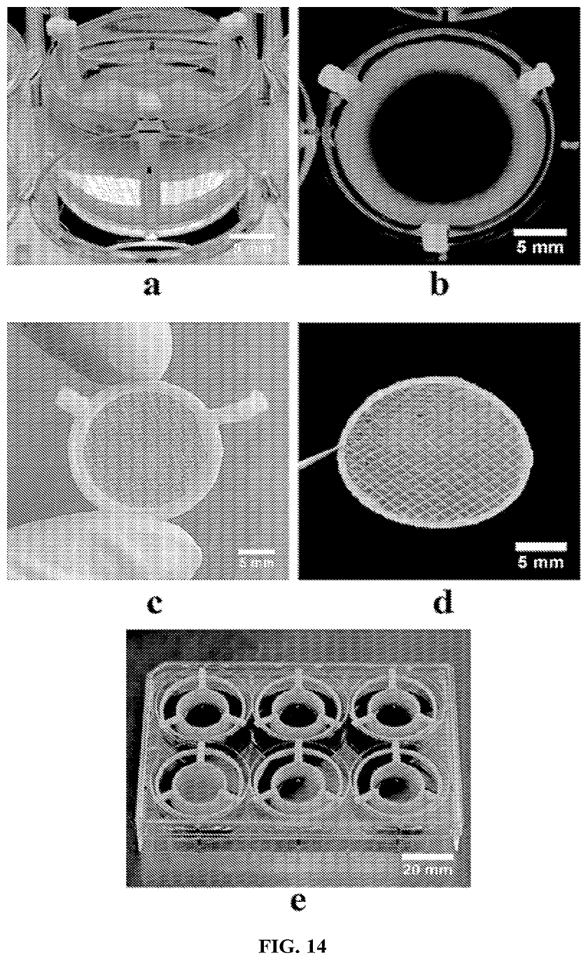

FIG. a and FIG. b in FIG. 14 show a front view and a top view of a physical object in the process of preparing a membranous cell sheet having a tight junction structure. FIG. c in FIG. 14 shows a physical image of the membranous cell sheet having a tight junction structure prepared and collected by a collection support. FIG. d in FIG. 14 shows a physical image of the membranous cell sheet on a mesh substrate support after the membranous cell sheet having a tight junction structure is prepared. From FIG. d in FIG. 14, it can be seen that the membranous cell sheet forms a tight junction with a macroscopic size and is completely attached to the substrate support. FIG. e in FIG. 14 is a physical image of an in vitro blood-brain barrier model.

### Characterization of a Product:

The tight junction structure of the endothelial membranous cell sheet in the embodiment is verified through micrographs and electron microscopy images. From FIG. 15, it can be seen that the membranous cell sheet forms a tight junction with a microscopic size and is completely attached to the substrate support.

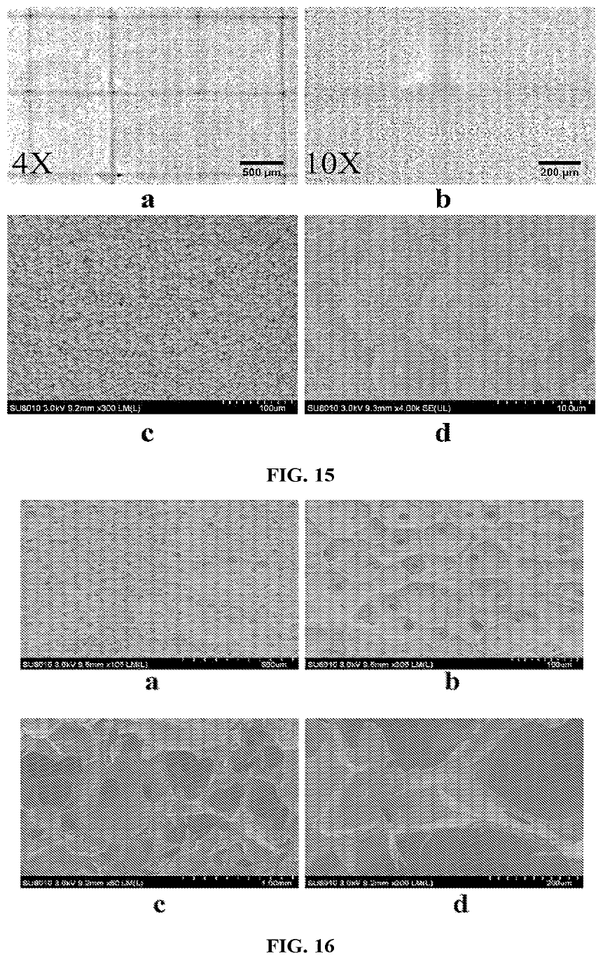

It is verified through electron microscopy images that the hydrogel used for encapsulation in the embodiment does not have a barrier function, and that is to say, the barrier function is achieved by the constructed membranous cell sheet. From FIG. 16, it can be seen that the hydrogel used for encapsulation in a microscopic size is of a loose and porous hollow structure, so that the passing of materials can be guaranteed.

A fluorescence permeation experiment is carried out to verify the edge tightness of the hydrogel used for encapsulation in the embodiment.

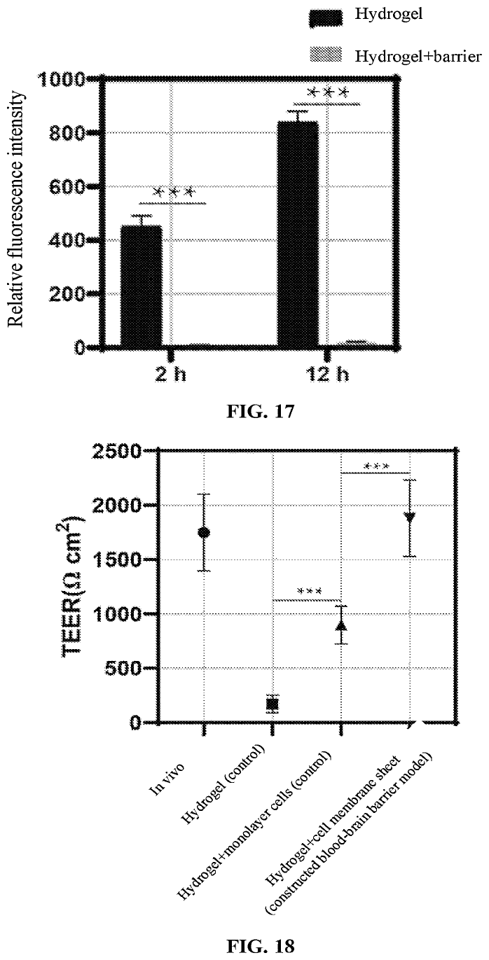

A specific experimental process is as follows: the bracket and the mesh substrate support loaded with the membranous cell sheet were separately encapsulated with the hydrogel with/without a barrier, then fluorescent dyes were added to the upper chamber separately, and after a period of time, the fluorescence intensity in the lower chamber was detected to reflect the leakage degree of the fluorescent dyes. Detection results are as shown in FIG. 17.

According to the results in FIG. 17, it can be seen that after 12 hours, the edge encapsulated with the hydrogel has no significant permeation, and has a good sealing property.

### Functional Tests of a Product:

Electrodes of a Millicell-ERS volt-ohmmeter transmembrane resistance measuring instrument were immersed in liquids (cell culture medium) in the upper chamber and the lower chamber respectively, and a transmembrane resistance test is carried out to determine differences in transmembrane resistance between the blood-brain barrier model constructed in the embodiment, a hydrogel control group, a hydrogel and monolayer cell control group, and an in vivo blood-brain barrier under the same conditions. Results are as shown in FIG. 18. According to the results in FIG. 18, it is shown that the transmembrane resistance of the blood-brain barrier model constructed in the embodiment (as shown as hydrogel and membranous cell sheet in the figure) is up to about 1,900 Ω/cm², which is similar to data of a human blood-brain barrier (in vivo), and is significantly different from that of the control group.

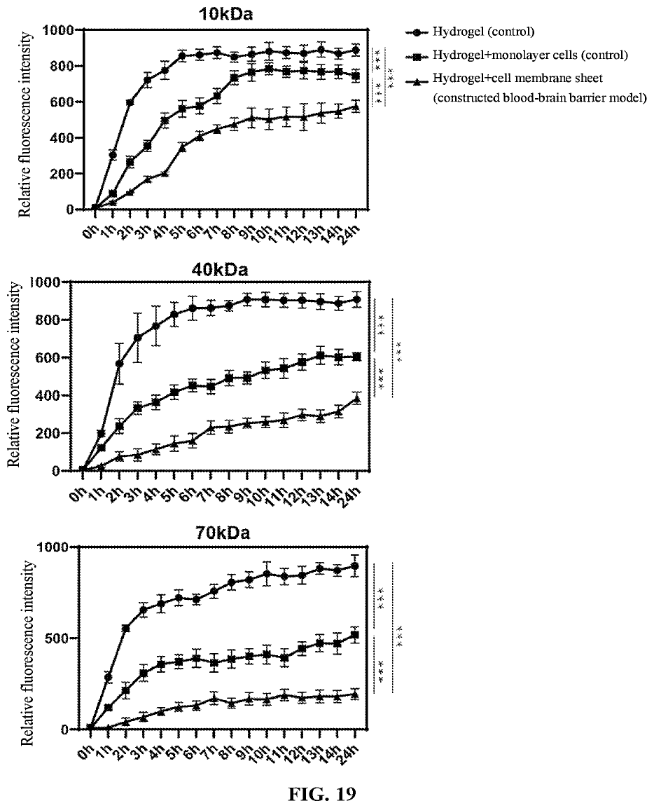

A permeation test of substances with different molecular weights is carried out to determine differences in permeability (barrier function) between the blood-brain barrier model constructed in the embodiment, a hydrogel control group and a hydrogel and monolayer cell control group under the same conditions. Results are as shown in FIG. 19. According to the results in FIG. 19, it is shown that the blood-brain barrier model constructed in the embodiment (as shown as hydrogel and membranous cell sheet in the figure) has a significant difference with the control group in permeability, namely, having a good barrier function.

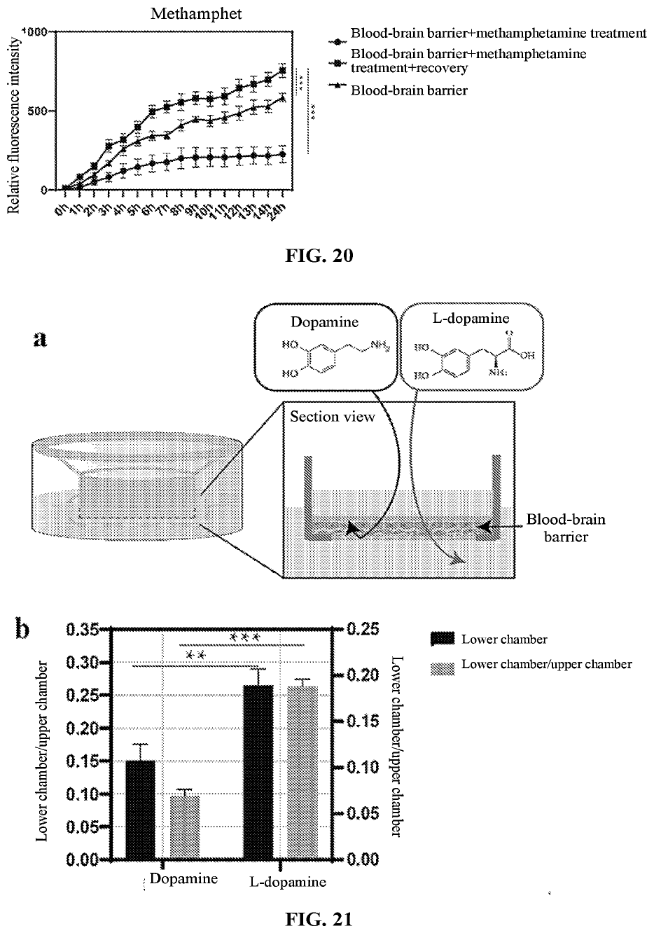

A test of a blood-brain barrier toxic drug is carried out to determine the response of the blood-brain barrier model constructed in the embodiment to the blood-brain barrier toxic drug. Results are as shown in FIG. 20. According to the results in FIG. 20, it is shown that the barrier function of the blood-brain barrier model constructed in the embodiment (as shown as blood-brain barrier in the figure) is significantly reduced after treatment with the blood-brain barrier toxic drug (methamphetamine). However, after self-recovery for 24 hours, the barrier function is significantly improved, which is consistent with response results of the human blood-brain barrier to the blood-brain barrier toxic drug (methamphetamine).

A permeation test of blood-brain barrier related drugs is carried out to determine the permeability (selective permeation function) of the blood-brain barrier model constructed in the embodiment to the blood-brain barrier related drugs. Results are as shown in FIG. 21. According to the results in FIG. 21, it is shown that after dopamine and L-dopamine are separately added to the upper chamber of the blood-brain barrier model constructed in the embodiment, the content of the L-dopamine detected in the lower chamber is significantly higher than that of the dopamine, indicating that the blood-brain barrier model constructed in the embodiment has a selective permeation function on the blood-brain barrier related drugs, which is consistent with selective permeation results of the blood-brain barrier related drugs by the human blood-brain barrier.

In summary, the in vitro blood-brain barrier model having a tight junction structure constructed by the present disclosure can well simulate functions of the human blood-brain barrier, so that the accuracy and effectiveness of the research and development of blood-brain barrier drugs are improved. Moreover, the culture method is simple to operate and easy to popularize, and an important research basis is provided for drug development and disease diagnosis and treatment of nervous system diseases.

The embodiments described above are merely preferred embodiments of the present disclosure. It should be pointed out that the above embodiments are exemplary and cannot be construed as limitations of the present disclosure. A variety of changes, modifications, substitutions, and variants can also be made by persons of ordinary skill in the technical field without departing from the principle of the present disclosure, and all the improvements and refinements shall also be deemed as the protection scope of the present disclosure.

1. A culture method of adherent cells generating a tight junction structure, comprising: inoculating adherent cells resuspended in a culture medium onto a bottom support liquid in a culture container, and completing culture on the surface of the bottom support liquid,
wherein the bottom support liquid has higher density than the culture medium, and is not miscible with the culture medium.

2. The culture method of adherent cells generating a tight junction structure according to claim 1, wherein the bottom support liquid is added to the culture container in advance, and the bottom support liquid is a mixture of one or more of fluorinated oil, fluoroalkane compounds, siloxane compounds, and ester compounds.

3. The culture method of adherent cells generating a tight junction structure according to claim 2, wherein the bottom support liquid is a mixture of one or more of fluorinated oil, silicone oil, uncured polydimethylsiloxane, dimethyl carbonate, and dimethyl sulfate.

4. The culture method of adherent cells generating a tight junction structure according to claim 1, wherein the bottom support liquid in the culture container is added in an amount of greater than 0.08 mL/cm².

5. The culture method of adherent cells generating a tight junction structure according to claim 1, wherein the adherent cells are inoculated in a concentration of 2*10₄-2*10⁸ pcs/cm².

6. The culture method of adherent cells generating a tight junction structure according to claim 1, wherein the adherent cells are cultured at a temperature of 35-39° C. for 1-28 days.

7. The culture method of adherent cells generating a tight junction structure according to claim 1, wherein the adherent cells are frozen cells after recovery or cells after passage digestion.

8. The culture method of adherent cells generating a tight junction structure according to claim 1, wherein the culture medium is replaced once every 10-15 hours during the culture, and the volume of the replaced culture medium is 70-90% of that of the original culture medium.

9. A membranous cell sheet having a tight junction structure, cultured by the culture method according to claim 1.

10. An in vitro blood-brain barrier model having a tight junction structure, comprising:
a tubular bracket;
a mesh substrate support for sealing the bottom of the bracket;
a membranous cell sheet that is loaded on the mesh substrate support and has a tight junction structure;
and a hydrogel structure for encapsulating the membranous cell sheet, the substrate support and the bracket as a whole.

11. The in vitro blood-brain barrier model having a tight junction structure according to claim 10, wherein the upper end of the bracket is provided with a positioning part for positioning the bracket.

12. The in vitro blood-brain barrier model having a tight junction structure according to claim 10, wherein the lower end of the bracket is provided with a supporting part for mounting the substrate support.

13. The in vitro blood-brain barrier model having a tight junction structure according to claim 10, wherein the hydrogel structure is prepared from a substance that is converted from a liquid state to a gel state after being stimulated by external conditions.

14. The in vitro blood-brain barrier model having a tight junction structure according to claim 13, wherein the hydrogel structure is prepared from one or more of gelatin, gelatin derivatives, hyaluronic acid, hyaluronic acid derivatives, alginate compounds, Pluronic F-127, fibrinogen, collagen, silk fibroin, chitosan, agarose, polyethylene glycol, and polyethylene oxide.

15. The in vitro blood-brain barrier model having a tight junction structure according to claim 10, wherein a culture method of the membranous cell sheet having a tight junction structure comprises:
inoculating cells resuspended in a culture medium onto a bottom support liquid in a culture container, and completing culture on the surface of the bottom support liquid to form the membranous cell sheet having a tight junction structure,
wherein the bottom support liquid has higher density than the culture medium, and is not miscible with the culture medium.

16. The in vitro blood-brain barrier model having a tight junction structure according to claim 15, wherein the bottom support liquid is a mixture of one or more of fluorinated oil, fluoroalkane compounds, siloxane compounds, and ester compounds;
and the cells are frozen cells after recovery or cells after passage digestion.

17. The in vitro blood-brain barrier model having a tight junction structure according to claim 15, wherein the cells are inoculated in a concentration of 2*10⁴-2*10⁸ pcs/cm² during cell culture.

18. The in vitro blood-brain barrier model having a tight junction structure according to claim 15, wherein the substrate support is buried in the bottom support liquid first, and then the cells are inoculated; after the membranous cell sheet is formed at the end of cell culture, the substrate support is lifted upward and removed, whereby the membranous cell sheet is attached to the substrate support, and the substrate support loaded with the membranous cell sheet is obtained.

19. A construction method of an in vitro blood-brain barrier model having a tight junction structure, comprising: preparing blood-brain barrier related endothelial cells into a membranous cell sheet having a tight junction structure, encapsulating the membranous cell sheet, an bracket with a customized size and a substrate support as a whole with a hydroge, and separating the bracket into an upper part and a lower part which are used for simulating an inner part and an outer part of a blood-brain barrier, so that physiological functions of the blood-brain barrier can be reproduced in vitro, and the permeability of a drug in the blood-brain barrier is observed.

20. Application of the in vitro blood-brain barrier model having a tight junction structure according to claim 10 to the research and development of blood-brain barrier related drugs.

21. The application according to claim 20, wherein the bottom of the blood-brain barrier model is suspended in a cell culture medium, a blood-brain barrier related drug is added to the cell culture medium inside the blood-brain barrier model, and the concentration of the drug in the cell culture medium outside the blood-brain barrier model is detected so as to determine a permeation effect of the drug in the blood-brain barrier model.
